(19) United States
(12) Patent Application Publication
(10) Pub. No.: US 2011/0097288 A1
(43) Pub. Date: Apr. 28, 2011

(54) Sun Protection Composition

(76) Inventors: Frank Janssen, Neuss (DE); Susanne Dickhof, Viersen (DE)

(21) Appl. No.: 12/910,886

(22) Filed: Oct. 25, 2010

(30) Foreign Application Priority Data
Oct. 26, 2009 (DE) 10 2009 045 998.7

(51) Int. Cl. A61K 8/18; A61Q 17/04

(52) U.S. Cl. 424/60; 424/59

(57) ABSTRACT

Cosmetic compositions with improved shelf stability for sun protecting factor. The compositions contain the UV filters 4-(tert-butyl)-4′-methoxydibenzoylmethane, Polysilicone-15, 2-cyano-3,3-phenylcinnamic acid 2-ethylhexyl ester, and at least one water-soluble UV filter, as well as at least one neutralization agent for the water-soluble UV filter, as well as at least one salt of C₁₂₋₂₀ alkyl phosphate, at least one C₁₄₋₂₀ mono- or diacylglyceride, the compositions having a pH of 6.0 to 8.0 at 20° C.

### CROSS REFERENCE TO RELATED APPLICATIONS

The present application claims priority to German Patent Application No. 10 2009 045 998.7-01 filed 26 Oct. 2009, which is incorporated herein by reference.

The present invention relates to cosmetic and dermatological compositions for protecting skin and hair from UV radiation.

The skin-damaging effect of the ultraviolet portion of solar radiation is known. UV radiation in the region from 290 nm to 320 nm (the ‘UV-B’ region) can lead to erythema, to simple sunburn, or to even more severe burns as acute phenomena. The narrow region around 308 nm is indicated as a maximum of the erythema effectiveness of sunlight. It has been incorrectly assumed for a long time that the longer-wave UV-A radiation, having a wavelength from 320 nm to 400 nm, has only a negligible biological effect. However, it has since been demonstrated by numerous studies that UV-A radiation is far more dangerous than UV-B radiation in initiating photodynamic, especially phototoxic reactions and chronic modifications of the skin. Accordingly, the damaging influence of UV-B radiation can also be further intensified by UV-A radiation. For example, it has been proven that under entirely normal everyday conditions, UV-A radiation itself is sufficient to damage within a short time collagen and elastin fibers that are important for the structure and strength of the skin. Chronic light-related skin changes occur as a result (i.e., the skin prematurely “ages”). The clinical appearance of skin aged by light includes, for example, folds and wrinkles as well an irregular, furrowed relief. In addition, those portions affected by light-related skin aging can exhibit irregular pigmentation. Formation of brown spots, keratoses, and even carcinomas or malignant melanomas is also possible. Skin prematurely aged by daily UV stress is further notable for lower Langerhans cell activity and a slight chronic inflammation.

Approximately 90% of the ultraviolet radiation striking the Earth is made up of UV-A rays. While the amount of UV-B radiation varies greatly as a function of numerous factors (e.g., season and time of day, or latitude), UV-A radiation remains relatively constant every day regardless of seasonal and diurnal or geographic factors. At the same time, most of UV-A radiation penetrates into the living epidermis, while approximately 70% of UV-B rays are held back by the stratum corneum.

It is therefore important that cosmetic and dermatological light protection compositions offer sufficient protection against both UV-B and UV-A radiation. A large number of cosmetic compositions containing at least one UV filter substance have already been developed for this purpose.

UV filter substances are typically present in liquid or crystalline form at room temperature and capable of absorbing ultraviolet rays and releasing the absorbed energy again in the form of longer-wave radiation (e.g., heat). UV-A filters and UV-B filters differ, depending on the wavelength region in which the absorption maximum of the filter lies.

It is often only with great difficulty that organic UV filters can be incorporated into cosmetic carriers in quantities required for a high light protection factor, wherein the compositions are stable over the long term and have pleasant, in particular non-sticky, properties.

It is advantageous for high-performance light protection compositions based on oil-in-water or water-in-oil (O/W or W/O) emulsions if at least one organic UV filter is present in both the oil phase and the water phase. Because UV-filters in major economic regions such as the USA, Europe, Japan or Australia are typically substances requiring regulatory approval, one skilled in the art of cosmetics is restricted to a relatively limited palette of approved UV filters for production of novel light protection compositions. A particularly preferred water-soluble organic UV filter is 2-phenylbenzimidazole-5-sulfonic acid. This compound, however, tends to crystallize out upon extended storage, causing the SPF (sun or light protection factor) of the composition to drop drastically.

Compositions in the existing art can be further improved. For example, compositions containing combinations of UV filter or other active substances occasionally present formulation-engineering problems with respect to utilization concentrations, making it difficult to achieve good UV protection performance.

A further disadvantage is that typical light protection formulations often leave behind a sticky film on the skin.

Accordingly, the present invention provides light protection compositions offering a high level of protection against the effects of radiation in the UV-A and UV-B region, in particular solar radiation, while at the same time having improved sensory properties. Compositions according to the present invention further provide good skin-care properties, particularly effectiveness against skin aging phenomena (e.g., wrinkling, loss of elasticity), as well as a pleasant skin feel and ease of distribution over the skin.

In addition, compositions according to the present invention provide a high light protection factor (SPF) with a low concentration of organic UV filters. Particularly preferably, an SPF of at least 30 is achieved.

In September 2006, a recommendation of the EU Commission (EU Commission Recommendation of 22 Sep. 2006 on the efficacy of sunscreen products and the claims made relating thereto (2006/647/EC)) was issued, requiring sun protection agents to offer a minimum level of UV-A protection. If the protection performance (UV-A protection factor) is a third that of the sun protection factor, the product can carry a new logo for sufficient UV-A protection (see EU Commission Recommendation 2006/647/EC, §3, ¶ 10b. In order to determine the UV-A protection factor, the EU recommendation permits in vivo methods, or in vitro methods that have been demonstrated to correlate with in vivo methods.

Accordingly, the present invention provides for a sun protection composition that meets the requirement of EU Commission Recommendation 2006/647/EC, §3, ¶ 10b and exhibits a UVA protection of UV-A protection factor of ⅓ of the sun protection factor:

$$\frac{{SPF}_{in vivo}}{UVAPF} < 3$$

wherein in vitro UVA protection factor (“UVAPF”) is defined as—

$$UVAPF = \frac{{\int }_{λ = 320 nm}^{λ = 400 nm} P ( λ ) * I ( λ ) * d λ}{{\int }_{λ = 320 nm}^{λ = 400 nm} P ( λ ) * I ( λ ) * {10}^{- A ( λ ) * C} * d λ}$$

where
P(λ)=PPD effect spectrum (PPD=persistent pigment darkening; see V. Wendel et al., The influence of pre-irradiation on the predictability of an in vivo UVA protection with a new in vitro method, Photodermatol. Photoimmunol. Photomed., Vol. 19, pp. 93-97)
I(λ)=Illumination intensity (spectral irradiance) of the UV source (UVA 320-400 nm for PPD test)
C=Correction factor, determined iteratively, to adapt the in-vitro SPF to the in-vivo SPF (see below); should have a value from 0.8 to 1.2
d(λ)=1 nm
A(λ)=average monochromatic absorption of the test product after UV irradiation.
   and wherein in vitro sun protection factor (“SPF_{in vitro}”) is defined as—

$${SPF}_{in vitro} = \frac{{\int }_{λ = 290 nm}^{λ = 400 nm} E ( λ ) * I ( λ ) * d λ}{{\int }_{λ = 290 nm}^{λ = 400 nm} E ( λ ) * I ( λ ) * {10}^{- {A}_{0} ( λ )} * d λ}$$

where
E(λ)=Erythema effect spectrum (CIE-1987)
I(λ)=Illumination intensity (spectral irradiance) of the UV source (sunlight spectrum for SPF test)
A₀(λ)=Average monochromatic absorption of the test product before UV irradiation
d(λ)=1 nm

$${SPF}_{i n vitro , adj} = SPF labelled = \frac{{\int }_{λ = 290 nm}^{λ = 400 nm} E ( λ ) * I ( λ ) * d λ}{{\int }_{λ = 290 nm}^{λ = 400 nm} E ( λ ) * I ( λ ) * {10}^{- {A}_{0} ( λ ) * C} * d λ}$$

SPF labeled=SPF_{in vivo}.

In vitro UVA protection factor before UV exposure is defined as follows—

$${UVAPF}_{0} = \frac{{\int }_{λ = 320 nm}^{λ = 400 nm} P ( λ ) * I ( λ ) * d λ}{{\int }_{λ = 320 nm}^{λ = 400 nm} P ( λ ) * I ( λ ) * {10}^{- {A}_{0} ( λ ) * C} * d λ}$$

UVA radiation sample dose “D” (required for the measurements in order to obtain a reasonable correlation between UVAPF in vitro and in vivo PPD (‘persistent pigment darkening’) values) is calculated according to Colipa (The European Cosmetics Association) recommendation as follows: D=UVAPF₀*1.2 J/cm².

The present invention further provides compositions having an SPF that is as stable as possible over the long term and in storage, wherein the initially measured SPF decreases as little as possible after multiple weeks of storage.

The present invention also provides, in shelf-stable form, light protection compositions containing at least one water-soluble organic UV filter.

These compositions are further defined by the subject matter of the claims of the present Application.

The subject matter of the present Application is directed towards cosmetic compositions in the form of an oil-in-water emulsion containing:
a) UV filter 4-(tert-butyl)-4′-methoxydibenzoylmethane,
b) UV filter Polysilicone-15,
c) UV filter 2-cyano-3,3-phenylcinnamic acid 2-ethylhexyl ester (octocrylene),
d) at least one water-soluble UV filter selected from at least one derivative of benzimidazole sulfonic acid,
e) at least one neutralization agent for the water-soluble UV filter chosen from amino-C₁ to C₆ alkanols and/or amino-C₁ to C₆ alkanediols, particularly 2-amino-2-methylpropan-1-ol, 2-amino-2-methylpropane-1,3-diol, 2-aminopropan-1-ol, 3-aminopropan-1-ol, 1-aminopropan-2-ol (MIPA), or 2-amino-2-(hydroxymethyl)propane-1,3-diol (TRIS),
f) at least one salt of C₁₂₋₂₀ alkyl phosphate, particularly a salt of cetyl phosphate, as an anionic oil-in-water emulsifier,
g) at least one C₁₄₋₂₀ mono- or diacylglyceride, preferably at least one C₁₆₋₁₈ mono- or diacylglyceride, particularly preferably chosen from hardened palm oil glycerides,
h) having a pH at 20° C. from 6.0 to 8.0.

Preferred cosmetic oil-in-water emulsions according to the present invention have a pH in the range from 6.5 to 7.5 at 20° C., preferably a pH in the range from 6.8 to 7.3.

Many consumers prefer oil-in-water emulsions to water-in-oil emulsions because of the more pleasant skin feel. Because the external water phase in such emulsions often has a higher weight proportion than the dispersed oil/fat phase, it is advantageous to distribute the UV filter substances over both phases, having at least one water-soluble UV filter substance in addition to the at least one oil-soluble one. Cosmetic oil-in-water emulsions according to the present invention therefore contain at least one water-soluble organic UV filter chosen from at least one derivative of benzimidazole sulfonic acid.

It has been found, surprisingly, that long-term stability of the initial SPF is considerably improved due to use of a neutralizing agent for the water-soluble UV filter chosen from amino-C₁ to C₆ alkanols and/or amino-C₁ to C₆ alkanediols, particularly 2-amino-2-methylpropan-1-ol, 2-amino-2-methylpropane-1,3-diol, 2-aminopropan-1-ol, 3-aminopropan-1-ol, 1-aminopropan-2-ol (MIPA), or 2-amino-2-(hydroxymethyl)propane-1,3-diol (TRIS).

According to the present invention, preferred salts of C₁₂₋₂₀ alkyl phosphates that form, as an anionic oil-in-water emulsifier, a first part of the O/W emulsifier system used according to the present invention include monoesters of phosphoric acid with lauryl alcohol, tridecyl alcohol, isotridecyl alcohol, myristyl alcohol, pentadecyl alcohol, cetyl alcohol, palmityl alcohol, isocetyl alcohol, isostearyl alcohol, stearyl alcohol, oleyl alcohol, elaidyl alcohol, linoleyl alcohol, linolenyl alcohol, nonadecyl alcohol, arachyl alcohol, gadoleyl alcohol, or arachidonyl alcohol, which are present as an alkali, alkaline earth, ammonium, alkylammonium, alkanolamine, or glucammonium salt, preferably the corresponding sodium, potassium, alkanolamine, trialkylammonium, triethanolamine, 2-amino-1-butanol, 2-amino-2-methyl-1-propanol, 2-amino-2-methyl-1,3-propanediol, 2-amino-2-ethyl-1,3-propanediol, or tris(hydroxy-methyl)aminomethane salts as well as salts of the basic amino acids ornithine, lysine, arginine, and/or histidine. Potassium salts of the aforesaid phosphoric acid monoesters are preferred. Dipotassium monocetyl phosphate is particularly preferred.

Further preferred salts of C₁₂₋₂₀ alkyl phosphates that form, as an anionic oil-in-water emulsifier, a first part of the O/W emulsifier system used according to the present invention include diesters of phosphoric acid with lauryl alcohol, tridecyl alcohol, isotridecyl alcohol, myristyl alcohol, pentadecyl alcohol, cetyl alcohol, palmityl alcohol, isocetyl alcohol, isostearyl alcohol, stearyl alcohol, oleyl alcohol, elaidyl alcohol, linoleyl alcohol, linolenyl alcohol, nonadecyl alcohol, arachyl alcohol, gadoleyl alcohol, or arachidonyl alcohol, which are present as an alkali, alkaline earth, ammonium, alkylammonium, alkanolamine, or glucammonium salt, preferably the corresponding sodium, potassium, alkanolamine, trialkylammonium, triethanolamine, 2-amino-1-butanol, 2-amino-2-methyl-1-propanol, 2-amino-2-methyl-1,3-propanediol, 2-amino-2-ethyl-1,3-propanediol, or tris(hydroxymethyl)aminomethane salts as well as salts of the basic amino acids ornithine, lysine, arginine, and/or histidine. Potassium salts of the aforesaid phosphoric acid diesters are preferred. Potassium dicetyl phosphate is particularly preferred. Mixtures of mono-C₁₂₋₂₀ alkyl phosphates and di-C₁₂₋₂₀ alkyl phosphates are particularly preferred. Mixtures of dipotassium monocetyl phosphate and potassium dicetyl phosphate are extraordinarily preferred.

Preferred C₁₄₋₂₀ mono- or diacylglycerides, which form the second part of the O/W emulsifier system, include monomyristoyl glyceride, monopalmitoyl glyceride, monostearoyl glyceride, monoarachinoyl glyceride, dimyristoyl glyceride, dipalmitoyl glyceride, distearoyl glyceride, and diarachinoyl glyceride. Further C₁₄₋₂₀ mono- or diacylglycerides include glycerides of hardened (i.e., hydrogenated), preferably completely hydrogenated, fatty acids of natural oils. Hardened palm oil glycerides are particularly preferred according to the present invention.

A particularly preferred O/W emulsifier system contains a mixture of dipotassium monocetyl phosphate and potassium dicetyl phosphate with hardened palm oil glycerides and is commercially obtainable from the Symrise company under the trade name “Emulsiphos 677660” (INCI: Potassium Cetyl Phosphate, Hydrogenated Palm Glycerides).

O/W emulsifier systems having at least one salt of C₁₂₋₂₀ alkyl phosphate as an anionic oil-in-water emulsifier and at least one C₁₄₋₂₀ mono- or diacylglyceride are present in an amount of from 0.5 wt % to 10 wt %, preferably 1 to 7 wt %, particularly 2.5 to 4.5 wt %, and more preferably 3 to 4 wt %, based on total weight of the composition.

Stabilization of the light protection factor (SPF) in the pH range 6.0 to 8.0 with at least one amino-C₁ to C₆ alkanol and/or at least one amino-C₁ to C₆ alkanediol, in combination with the at least one anionic oil-in-water emulsifier chosen from the salts of C₁₂₋₂₀ alkyl phosphate, and the at least one C₁₄₋₂₀ mono- or diacylglyceride, was observed in particular when 4-(tert-butyl)-4′-methoxydibenzoylmethane, Polysilicone-15, 2-cyano-3,3-phenylcinnamic acid 2-ethylhexyl ester, and at least one water-soluble UV filter chosen from at least one derivative of benzimidazole sulfonic acid are present.

4-(tert-butyl)-4′-methoxydibenzoylmethane belongs to the alkyl- and/or alkoxy-substituted dibenzoylmethane derivatives representing oil-soluble organic UV-A filters. 4-tert-butyl-4′-methoxydibenzoylmethane, having the INCI designation Butyl Methoxydibenzoylmethane (also referred to as 1-(4′-tert-butylphenyl)-3-(4′-methoxyphenyl)propane-1,3-dione), is obtainable, for example, as Parsol® 1789 from DSM or as Eusolex® 9020 from Merck KGaA.

Preferred oil-in-water emulsions according to the present invention contain 4-(tert-butyl)-4′-methoxydibenzoylmethane in an amount of from 0.5 wt % to 20 wt %, preferably 1 to 15 wt %, particularly preferably 2 to 10 wt %, and extraordinarily preferably 3 to 5 wt %, based on total weight of the composition.

Polysilicone-15 (INCI name) is also referred to as 3-(4-(2,2-bis-ethoxycarbonylvinyl)phenoxy)propenyl)methoxysiloxane/dimethylsiloxane copolymer with Parsol® SLX), dimethicodiethylbenzalmalonate, diethylbenzylidene malonate dimethicone, or diethylmalonylbenzylidene oxypropene dimethicone, and is commercially obtainable as Parsol® SLX (INCI name Dimethicodiethylbenzal Malonate (CAS no. 207574-74-1)) from DSM. The chemical structure is described, for example, in EP 0 709 080 A2.

Polysilicone-15 is present in an amount of from 0.5 wt % to 10 wt %, preferably 1 to 7 wt %, particularly 1.5 to 5 wt %, and more preferably 2 to 4 wt %, based on total weight of the composition.

2-Cyano-3,3-phenylcinnamic acid 2-ethylhexyl ester (=ethylhexyl-2-cyano-3,3-diphenylacrylate (INCI: Octocrylene)) is commercially available under the names Parsol® 340, Eusolex® OCR, Uvinul® N 539, and Neo Heliopan® 303.

Octocrylene is preferably present in an amount of from 0.5 wt % to 15 wt %, particularly preferably 1 to 7 wt %, more particularly 2 to 5 wt %, and even more preferably 3 to 4 wt %, based on total weight of the composition.

Preferred cosmetic oil-in-water emulsions according to the present invention contain at least one water-soluble organic UV filter chosen from at least one derivative of benzimidazole sulfonic acid in an amount of from 0.5 wt % to 15 wt %, preferably 1 to 10 wt %, more preferably 2 to 5 wt %, and even more preferably 3 to 4 wt %, based on total weight of the composition.

### Derivative of Benzimidazole Sulfonic Acid

According to the present invention, part of the at least one benzimidazole sulfonic acid derivative is present as a salt of at least one amino-C₁ to C₆ alkanol and/or at least one amino-C₁ to C₆ alkanediol. This is equivalent to at least 30 mol %, preferably at least 50 mol %, and more preferably at least 70 mol %, based on total content of benzimidazole sulfonic acid derivative. Preferred amino-C₁ to C₆ alkanols and amino-C₁ to C₆ alkanediols include 2-amino-2-methylpropan-1-ol, 2-amino-2-methylpropane-1,3-diol, 2-aminopropan-1-ol, 3-aminopropan-1-ol, 1-aminopropan-2-ol (MIPA), and 2-amino-2-(hydroxymethyl)propane-1,3-diol (TRIS), with 2-amino-2-methylpropan-1-ol and 2-amino-2-methylpropane-1,3-diol particularly preferred.

In addition to the salt of at least one amino-C₁ to C₆ alkanol and/or at least one amino-C₁ to C₆ alkanediol, further salts of the at least one benzimidazole sulfonic acid derivative can be present. Alkali, alkaline earth, ammonium, alkylammonium, alkanolamine, and glucammonium salts are preferred, preferably the corresponding sodium, potassium, trialkylammonium, or triethanolammonium salts.

Derivatives of benzimidazole sulfonic acid preferred according to the present invention as water-soluble UV filter substance(s) include phenylene-1,4-bis(2-benzimidazyl)-3,3′-5,5′-tetrasulfonic acid (UV-A) and its salts.

Phenylene-1,4-bis-(2-benzimidazyl)-3,3′-5,5′-tetrasulfonic acid bis-sodium salt, having the INCI name Disodium Phenyl Dibenzimidazole Tetrasulfonate (CAS no.: 180898-37-7) and commercially available from Symrise as Neo Heliopan AP is preferred. Further preferred water-soluble organic UV filter substances include 2-phenylbenzimidazole-5-sulfonic acid (INCI name Phenylbenzimidazole Sulfonic Acid (CAS no. 27503-81-7), commercially available under the names Neo Heliopan Hydro from Symrise or Eusolex 232 from Merck KGaA; UV-B filter) and its salts.

The statements made above apply to compositions of the salts.

In a particularly preferred embodiment of the invention, the at least one water-soluble organic UV filter representing a derivative of benzimidazole sulfonic acid is chosen from phenylene-1,4-bis-(2-benzimidazyl)-3,3,1-5,5′-tetrasulfonic acid, 2-phenylbenzimidazole-5-sulfonic acid, and the salts of said acids with 2-amino-2-methylpropan-1-ol or 2-amino-2-methylpropane-1,3-diol, particularly the salts of phenylbenzimidazole sulfonic acid and of phenylene-1,4-bis-(2-benzimidazyl)-3,3′-5,5′-tetrasulfonic acid bis-sodium salt with 2-amino-2-methylpropan-1-ol or 2-amino-2-methylpropane-1,3-diol as counterions. In a particularly preferred embodiment, these preferred salts can be present in combination with alkali, alkaline earth, ammonium, alkylammonium, alkanolamine, and glucammonium salts, preferably with sodium, potassium, trialkylammonium, or triethanolamine salts.

Preferred oil-in-water emulsions according to the present invention contain derivative(s) of benzimidazole sulfonic acid in an amount of from 0.5 wt % to 15 wt %, preferably 1 to 10 wt %, more preferably 2 to 5 wt %, and even more preferably 3 to 4 wt %, based on total weight of the composition.

Regardless of the form in which the benzimidazole sulfonic acid derivative is present (e.g., salt, free acid), weight refers to the weight of the benzimidazole sulfonic acid derivative in its acid form.

Further preferred oil-in-water emulsions contain 2-phenylbenzimidazole-5-sulfonic acid or a salt thereof in an amount of from 0.5 wt % to 15 wt %, preferably 1 to 10 wt %, more preferably 2 to 5 wt %, and even more preferably 3 to 4 wt %, based on total weight of the composition.

Further preferred oil-in-water emulsions contain phenylene-1,4-bis(2-benzimidazyl)-3,3′-5,5′-tetrasulfonic acid or a salt thereof in an amount of from 0.5 wt % to 15 wt %, preferably 1 to 10 wt %, more preferably 2 to 5 wt %, and even more preferably 3 to 4 wt %, based on total weight of the composition, with the total amount of all benzimidazole sulfonic acid derivatives being from 0.5 wt % to 15 wt %, preferably 1 to 10 wt %, more preferably 2 to 5 wt %, and even more preferably 3 to 4 wt %.

Further preferred oil-in-water emulsions contain both phenylene-1,4-bis(2-benzimidazyl)-3,3′-5,5′-tetrasulfonic acid and 2-phenylbenzimidazole-5-sulfonic acid, respectively in acid and/or salt form, in an amount of from 0.5 wt % to 15 wt %, preferably 1 to 10 wt %, more preferably 2 to 5 wt %, and even more preferably 3 to 4 wt %, based on total weight of the composition according to the present invention, with the total amount of all benzimidazole sulfonic acid derivatives being from 0.5 wt % to 15 wt %, preferably 1 to 10 wt %, more preferably 2 to 5 wt %, and even more preferably 3 to 4 wt %.

Particularly preferred oil-in-water emulsions contain phenylene-1,4-bis(2-benzimidazyl)-3,3′-5,5′-tetrasulfonic acid and 2-phenylbenzimidazole-5-sulfonic acid in a weight ratio of 0.2 to 1, preferably 0.3 to 0.8, and more preferably 0.4 to 0.5.

According to manufacturer information from WO 2007/135196 A2, derivatives of benzimidazole sulfonic acid can be stabilized with regard to crystallization by neutralization using basic amino acids, in particular, using ornithine, lysine, arginine, and/or histidine, with arginine being particularly preferred. It was not possible to confirm this for the subject matter of the present Application.

In a preferred embodiment, oil-in-water emulsions according to the present invention contain basic amino acids such as lysine, arginine, and histidine in an amount of from 0 to at most 0.1 wt %, based on total composition.

Further preferred oil-in-water emulsions according to the present invention contain a) 4-(tert-butyl)-4′-methoxydibenzoylmethane, b) Polysilicone-15, c) 2-cyano-3,3-phenylcinnamic acid 2-ethylhexyl ester, and d) 2-phenylbenzimidazole-5-sulfonic acid (salt).

Further preferred oil-in-water emulsions contain a) 4-tert-butyl-4′-methoxydibenzoylmethane, b) Polysilicone-15, c) 2-cyano-3,3-phenylcinnamic acid 2-ethylhexyl ester, and d) disodium phenylbenzimidazole tetrasulfonate.

Further preferred oil-in-water emulsions according to the present invention contain UV filters a), b), c), and d) in a weight ratio a):b):c):d) to one another of (2.7 to 3.3):(1.8 to 2.2):(3.5 to 4.5):(2.7 to 3.3).

More preferably, the UV filters a), b), c), and d) are present in a weight ratio a):b):c):d) to one another of (2.7 to 3.3):(1.8 to 2.2):(1.8 to 3.3):(2.7 to 3.3), even more preferably in a weight ratio a):b):c):d) to one another of (2.7 to 3.3):(1.8 to 2.2):(1.8 to 2.2):(2.7 to 3.3).

Further preferred oil-in-water emulsions according to the present invention contain a mixture of dipotassium monocetyl phosphate and potassium dicetyl phosphate with hardened palm oil glycerides, furthermore 4-tert-butyl-4′-methoxydibenzoylmethane, Polysilicone-15, 2-cyano-3,3-phenylcinnamic acid 2-ethylhexyl ester, and the sodium salt of 2-phenylbenzimidazole-5-sulfonic acid, and have a pH at 20° C. from 6.0 to 8, preferably 6.5 to 7.5, and more preferably 6.8 to 7.3.

Further preferred oil-in-water emulsions according to the present invention contain a mixture of dipotassium monocetyl phosphate and potassium dicetyl phosphate with hardened palm oil glycerides, furthermore 4-tert-butyl-4′-methoxydibenzoylmethane, Polysilicone-15, 2-cyano-3,3-phenylcinnamic acid 2-ethylhexyl ester, and the AMP salt (2-amino-2-methylpropan-1-ol salt) of 2-phenylbenzimidazole-5-sulfonic acid, and have a pH at 20° C. from 6.0 to 8, preferably 6.5 to 7.5, and more preferably 6.8 to 7.3.

Further preferred oil-in-water emulsions contain a mixture of dipotassium monocetyl phosphate and potassium dicetyl phosphate with hardened palm oil glycerides in an amount of from 0.5 wt % to 10 wt %, preferably 1 to 7 wt %, more preferably 2.5 to 4.5 wt %, and even more preferably 3 to 4 wt %. In one aspect, the emulsions contain Polysilicone-15 in an amount of from 0.5 wt % to 10 wt %, preferably 1 to 7 wt %, more preferably 2 to 5 wt %, and even more preferably 3 to 4 wt %; 4-tert-butyl-4′-methoxydibenzoylmethane in an amount of from 0.5 wt % to 20 wt %, preferably 1 to 15 wt %, more preferably 2 to 10 wt %, and even more preferably 3 to 5 wt %, and the sodium salt of 2-phenylbenzimidazole-5-sulfonic acid in an amount of from 0.5 wt % to 15 wt %, preferably 1 to 10 wt %, more preferably 2 to 5 wt %, and even more preferably 3 to 4 wt %, based on total weight of the composition, and having a pH at 20° C. from 6.0 to 8, preferably 6.5 to 7.5, more preferably 6.8 to 7.3.

Further preferred oil-in-water emulsions contain a mixture of dipotassium monocetyl phosphate and potassium dicetyl phosphate with hardened palm oil glycerides; furthermore, Polysilicone-15, 4-tert-butyl-4′-methoxydibenzoylmethane, the sodium salt and the 2-amino-2-methylpropan-1-ol salt of 2-phenylbenzimidazole-5-sulfonic acid, and have a pH at 20° C. from 6.0 to 8, preferably 6.5 to 7.5, particularly preferably 6.8 to 7.3.

Further preferred oil-in-water emulsions contain a mixture of dipotassium monocetyl phosphate and potassium dicetyl phosphate with hardened palm oil glycerides in an amount of from 0.5 to 10 wt %, preferably 1 to 7 wt %, more preferably 2.5 to 4.5 wt % and even more preferably 3 to 4 wt %; furthermore Polysilicone-15 in an amount of from 0.5 to 10 wt %, preferably 1 to 7 wt %, more preferably 2 to 5 wt %, and even more preferably 3 to 4 wt %; 4-tert-butyl-4′-methoxydibenzoylmethane in an amount of from 0.5 to 20 wt %, preferably 1 to 15 wt %, more preferably 2 to 10 wt %, and even more preferably 3 to 5 wt %; 2-cyano-3,3-phenylcinnamic acid 2-ethylhexyl ester in an amount of from 0.5 to 15 wt %, preferably 1 to 7 wt %, more preferably 2 to 5 wt %, and even more preferably 3 to 4 wt %; the sodium salt and the 2-amino-2-methylpropan-1-ol salt of 2-phenylbenzimidazole-5-sulfonic acid in an amount of from 0.5 to 15 wt %, preferably 1 to 10 wt %, more preferably 2 to 5 wt %, and even more preferably 3 to 4 wt %, based on total weight of the composition, and having a pH at 20° C. from 6.0 to 8, preferably 6.5 to 7.5, more preferably 6.8 to 7.3.

Further preferred oil-in-water emulsions contain a mixture of dipotassium monocetyl phosphate and potassium dicetyl phosphate with hardened palm oil glycerides in an amount of from 0.5 to 10 wt %, preferably 1 to 7 wt %, more preferably 2.5 to 4.5 wt %, and even more preferably 3 to 4 wt %; furthermore, Polysilicone-15 in an amount of from 0.5 to 10 wt %, preferably 1 to 7 wt %, more preferably 2 to 5 wt %, and even more preferably 3 to 4 wt %; 4-tert-butyl-4′-methoxydibenzoylmethane in an amount of from 0.5 to 20 wt %, preferably 1 to 15 wt %, more preferably 2 to 10 wt % and even more preferably 3 to 5 wt %; 2-cyano-3,3-phenylcinnamic acid 2-ethylhexyl ester in an amount of from 0.5 to 15 wt %, preferably 1 to 7 wt %, more preferably 2 to 5 wt %, and even more preferably 3 to 4 wt %; respectively, the sodium salt and the 2-amino-2-methylpropan-1-ol salt of 2-phenylbenzimidazole-5-sulfonic acid and phenylene-1,4-bis-(2-benzimidazyl)-3,3′-5,5′-tetrasulfonic acid in an amount of from 0.5 to 15 wt %, preferably 1 to 10 wt %, more preferably 2 to 5 wt %, and even more preferably 3 to 4 wt %, based on total weight of the composition, and having a pH at 20° C. of from 6.0 to 8, preferably 6.5 to 7.5, more preferably 6.8 to 7.3.

In order to increase the light protection factor further, compositions according to the present invention can contain at least one further organic and/or inorganic UV filter substance.

### S-Triazine Derivatives

It has been found, surprisingly, that with an O/W emulsifier system containing at least one salt of C₁₂₋₂₀ alkyl phosphate and at least one C₁₄₋₂₀ mono- or diacylglyceride in the mentioned pH range, lower SPF values and less shelf stability can be obtained in some cases with oil-soluble organic UV-filter substances based on s-triazine derivatives having the following structural element—

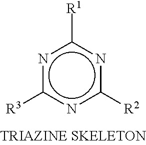

than with, for example, Polysilicone-15.

In a preferred embodiment, oil-in-water emulsions according to the present invention are therefore free of s-triazine derivatives having the above TRIAZINE SKELETON structural element.

s-Triazine derivatives having the above TRIAZINE SKELETON structural element are known in the existing art (e.g., from EP 0 775 698, EP 0 878 469, and EP 1 027 781). Both symmetrical substitution and asymmetrical substitution are conceivable with regard to the C₃ axis of the triazine base element of these compounds. s-Triazines symmetrically substituted in this sense have three identical substituents R¹, R², and R³, whereas asymmetrically substituted s-triazine derivatives consequently have different substituents, with the result that the C₃ symmetry is destroyed. For purposes of the present invention, “asymmetrical” is always understood as asymmetrical in terms of the C₃ axis of the triazine base element unless something different is expressly mentioned.

Triazine derivatives that are symmetrical in terms of the C₃ axis of the triazine base element include those of the general formula TRIAZINE SKELETON wherein R¹═R²═R³=—NH-Phe-COOR, where Phe=phenyl residue in which the substituents —NH and —COOR are in the para-position with respect to one another. Corresponding symmetrical triazine derivatives include 4,4′,4″-(1,3,5-triazine-2,4,6-triyltriimino)-trisbenzoic acid-tris(alkyl esters), particularly 4,4′,4″-(1,3,5-triazine-2,4,6-triyltriimino)-trisbenzoic acid-tris(2-ethylhexyl ester) (INCI: Ethylhexyl Triazone, formerly Octyl Triazone), marketed as an individual substance by BASF under the commercial name UVINUL® T 150 but also obtainable in a variety of commercial UV filter mixtures.

Asymmetrical triazine derivatives include those disclosed in EP 0 775 698—

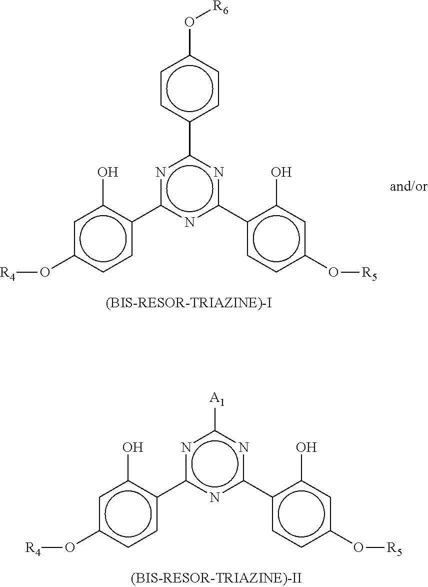

R₄ and R₅ can be chosen from branched and unbranched alkyl groups having 1 to 18 carbon atoms. The alkyl groups can also be substituted with silyloxyl groups.

A₁ represents, for example, a substituted homo- or heterocyclic aromatic five- or six-membered ring.

In the general formula (BIS-RESOR-TRIAZINE)-I, R₆ can be a hydrogen atom or a branched or unbranched alkyl group having 1 to 10 carbon atoms. An example of a compound of the general formula (BIS-RESOR-TRIAZINE)-I wherein R₄ and R₅ are each a 2-ethylhexyl group and R₆ is a methyl group is 2,4-bis-{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), commercially available from CIBA under the name Tinosorb® S.

A further example of an asymmetrically substituted s-triazine compound is a compound having the INCI name Diethylhexyl Butamido Triazone, having the general formula TRIAZINE SKELETON wherein R¹ and R² are —NH-Phe-COOR, where Phe=phenyl residue in which the —NH and —COOR substituents are in the para-position with respect to one another, R=2-ethylhexyl and R³=—NH-Phe-CONH-tert-butyl, where Phe=phenyl residue, in which the —NH and —CONH-tert-butyl substituents are in the para-position with respect to one another. This UV filter substance, a UV-A filter, is obtained from Sigma 3V under the commercial designation UVASORB HEB.

Further UV filter substances based on s-triazine compounds include—
2,4,6-tris([1,1′-biphenyl]-4-yl)-1,3,5-triazine, INCI: Tris-Biphenyl Triazine, commercially available from CIBA under the commercial name Tinosorb A2B;
2,4-bis-[5-1(di-methylpropyl)benzoxazol-2-yl-(4-phenyl)imino]-6-(2-ethylhexyl)imino-1,3,5-triazine (CAS no. 288254-16-0, Uvasorb® K2A of 3V Sigma, INCI: Ethylhexyl Bis-Isopentylbenzoxazolylphenyl Melamine);
2,4-bis-{[4-(3-sulfonato)-2-hydroxypropyloxy)-2-hydroxy]phenyl}-6-(4-methoxyphenyl)-1,3,5-triazine sodium salt;
2,4-bis-{[4-(3-(2-propyloxy)-2-hydroxypropyloxy)-2-hydroxy]phenyl}-6-(4-methoxyphenyl)-1,3,5-triazine;
2,4-bis-{[4-(2-ethylhexyloxy)-2-hydroxy]phenyl}-6-[4-(2-methoxyethylcarboxyl)phenyl-amino]-1,3,5-triazine;
2,4-bis-{[4-(3-(2-propyloxy)-2-hydroxypropyloxy)-2-hydroxy]phenyl}-6-[4-(ethylcarboxyl)phenylamino]-1,3,5-triazine;
2,4-bis-{[4-(2-ethylhexyloxy)-2-hydroxy]phenyl}-6-(1-methylpyrrol-2-yl)-1,3,5-triazine;
2,4-bis-{[4-tris(trimethylsiloxysilylpropyloxy)-2-hydroxy]phenyl}-6-(4-methoxyphenyl)-1,3,5-triazine;
2,4-bis-([4-(2-methylpropenyloxy)-2-hydroxy]phenyl}-6-(4-methoxyphenyl)-1,3,5-triazine;
2,4-bis-{[4-(1′,1′,1′,3′,5′,5′,5′-heptamethylsiloxy-2-methylpropyloxy)-2-hydroxy]phenyl}-6-(4-methoxyphenyl)-1,3,5-triazine, and
2-[4,6-bis(2,4-dimethylphenyl)-1,3,5-triazin-2-yl]-5-(ethylhexyloxy)phenol.

In order to increase the light protection factor further, compositions according to the present invention can contain at least one further organic and/or inorganic UV filter substance, which preferably differs from s-triazine derivatives according to the above TRIAZINE SKELETON structural element.

Further preferred oil-in-water emulsions contain a mixture of dipotassium monocetyl phosphate and potassium dicetyl phosphate with hardened palm oil glycerides; furthermore Polysilicone-15, 4-tert-butyl-4′-methoxydibenzoylmethane, and the sodium salt of 2-phenylbenzimidazole-5-sulfonic acid, having a pH at 20° C. of from 6.0 to 8, preferably 6.5 to 7.5, more preferably 6.8 to 7.3, and are free of s-triazine derivatives according to the above TRIAZINE SKELETON structural element.

Further advantageous oil-soluble organic UV filter substances that can be used in compositions according to the present invention are recited below.

### Benzotriazoles

One such preferred UV filter substance is 2,2′-methylene-bis-(6-(2H-benzotriazol-2-yl)-4-(1,1,3,3-tetramethylbutyl)phenol) (INCI: Methylene Bis-Benzotriazolyl Tetramethylbutyl-phenol, MBBT), having the following structural formula—

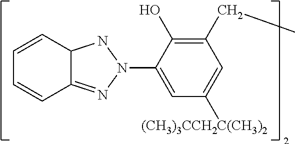

obtainable from CIBA under the tradename Tinosorb® M. This is a broadband filter that absorbs both UV-A and UV-B radiation.

Another optional broadband UV filter substance is 2-(2H-benzotriazol-2-yl)-4-methyl-6-[2-methyl-3-[1,3,3,3-tetramethyl-1-((trimethylsilyl)oxy]disiloxanyl]propyl]phenol (CAS no.: 155633-54-8) having the INCI designation Drometrizole Trisiloxane.

Further preferred optional benzotriazole filters include 2,2′-methyl-bis-[6(2H-benzotriazol-2-yl)-4-(methyl)phenol] (MIXXIM BB/200 from the Fairmount Chemical company), 2-(2′-hydroxy-3′,5′-di-t-amylphenyl)benzotriazole (CAS no.: 025973-551), 2-(2′-hydroxy-5′-octylphenyl)benzotriazole (CAS no. 003147-75-9), 2-(2′-hydroxy-5′-methylphenyl)benzotriazole (CAS no. 2440-22-4), and 2-(2H-benzotriazol-2-yl)-4-methyl-6-[2-methyl-3-[1,3,3,3-tetramethyl-1-((trimethylsilyl)oxy]disiloxanyl)propyl]phenol (CAS no.: 155633-54-8) with the INCI designation Drometrizole Trisiloxane.

Further preferred UV filter substances that can be used in compositions according to the present invention together with components (a), (b), (c), and (d) are illustrated below—
camphor derivatives, particularly 3-benzylidene camphor derivatives having no ionizable functional groups in the molecule, preferably 3-(4′-methylbenzylidene)-D,L camphor (INCI: 4-Methylbenzylidene Camphor), marketed by Merck under the trade name Eusolex 6300;
camphor derivatives having ionizable functional groups in the molecule, preferably sulfonic acid derivatives of 3-benzylidene camphor (e.g., 4-(2-oxo-3-bornylidene methyl)benzene sulfonic acid, 2-methyl-5-(2-oxo-3-bornylidene methyl)sulfonic acid and salts thereof; 1,4-di(2-oxo-10-sulfo-3-bornylidene methyl)benzene and salts thereof (especially the 10-sulfato compounds, particularly its corresponding salts, the statements made above for the derivatives of benzimidazole acid d) being applicable to the salts; this is also referred to as benzene-1,4-di(2-oxo-3-bornylidene methyl-10-sulfonic acid), sulfonic acid derivatives of 3-benzylidene camphor such as 4-(2-oxo-3-bornylidene methyl)benzenesulfonic acid and 2-methyl-5-(2-oxo-3-bornylidene)sulfonic acid and salts thereof, having the INCI designation Terephthalylidene Dicamphor Sulfonic Acid (CAS no. 92761-26-7, obtainable as Mexoryl SX from the Chimex company));
4-aminobenzoic acid derivatives, preferably 4-(dimethylamino)benzoic acid (2-ethylhexyl)-ester, 4-(dimethylamino)benzoic acid amyl ester;
2-aminobenzoic acid derivatives;
esters of cinnamic acid, preferably 4-methoxycinnamic acid (2-ethylhexyl) ester, 4-methoxycinnamic acid propyl ester, 4-methoxycinnamic acid isopentyl ester;
esters of salicylic acid, preferably salicylic acid (2-ethylhexyl) ester (2-ethylhexyl salicylate (=octyl salicylate)), salicylic acid (4-isopropylbenzyl) ester (4-isopropylbenzyl salicylate), salicylic acid homomethyl ester (homomethyl salicylate, homosalate);
benzophenone derivatives having no ionizable functional groups in the molecule, preferably 2-hydroxy-4-methoxy-4′-methylbenzophenone, 2,2′-dihydroxy-4-methoxybenzophenone, 2-hydroxy-4-methoxybenzophenone, 2-hydroxy-4-methoxy-4′-methylbenzophenone, 2,2′-dihydroxy-4-methoxybenzophenone; 2-(4′-diethylamino-2′-hydroxybenzoyl)benzoic acid-hexyl ester (also: aminobenzophenone, obtainable under the designation Uvinul A Plus from the BASF company);
benzophenone derivatives having ionizable functional groups in the molecule, preferably sulfonic acid derivatives of benzophenones, particularly preferably 2-hydroxy-4-methoxybenzophenone-5-sulfonic acid and salts thereof;
esters of benzalmalonic acid, preferably 4-methoxybenzalmalonic acid di(2-ethylhexyl) ester;
UV filters, other than Polysilicone-15, that are bound to polymers; and
Derivatives of benzoxazole, preferably 2,2′(naphthalene-1,4-diyl)bis(benzoxazole) (INCI: Dibenzoxazoyl Naphthalene) and 2,4-bis-[5-1(di-methylpropyl)benzoxazol-2-yl-(4-phenyl)imino]-6-(2-ethylhexyl)imino-1,3,5-triazine (Uvasorb® K2A of 3V Sigma, INCI: Ethylhexyl Bis-Isopentylbenzoxazolylphenyl Melamine).
   Benzoxazole derivatives are preferably present in dissolved form in cosmetic compositions according to the present invention. It may also be advantageous if, however, the benzoxazole derivatives are present in pigment form (i.e., undissolved), for example, at particle sizes from 10 nm to 300 nm.

Some of the oil-soluble UV filters can also serve as solvents or solubilizers for other UV filters. For example, solutions of the UV-A filter 1-(4-tert-butylphenyl)-3-(4′-methoxyphenyl)propane-1,3-dione (e.g., Parsol® 1789) in a variety of UV-B filters can be produced Compositions according to the present invention can therefore contain 1-(4-tert-butylphenyl)-3-(4′-methoxyphenyl)propane-1,3-dione in combination with at least one UV-B filter chosen from 4-methoxycinnamic acid 2-ethylhexyl ester, 2-cyano-3,3-phenylcinnamic acid 2-ethylhexyl ester, salicylic acid 2-ethylhexyl ester, and 3,3,5-trimethylcyclohexyl salicylate. In these combinations, the weight ratio of UV-B filter to the 1-(4-tert-butylphenyl)-3-(4′-methoxyphenyl)propane-1,3-dione is in a range of from 0.5 to 6, preferably from 0.6 to 3, with the molar ratio correspondingly in a range of from 0.3 to 3, preferably from 0.5 to 1.5.

The optional UV filter substances useful in the present invention and recited herein are of course not intended to be limiting.

Total amount of the at least one oil-soluble organic UV filter substance in cosmetic or dermatological compositions according to the invention is preferably 0.5 to 20 wt %, more preferably 1 to 15 wt %, and even more preferably 2 to 10 wt %, based on total weight of the composition.

Useful inorganic UV filter substances include finely dispersed or colloidally dispersed metal oxides and metal salts such as titanium dioxide, zinc oxide, iron oxide, aluminum oxide, cerium oxide, zirconium oxide, silicates (talc), and barium sulfate. Titanium dioxide and zinc oxide are particularly preferred. The particles should have an average diameter of about 100 nm or less, preferably from 5 to 50 nm, and more preferably from 15 to 30 nm. They can be spherical in shape, but it is also possible to use particles having an ellipsoidal shape or otherwise deviates from a spherical conformation. These particles can also be present in surface-treated form (i.e., hydrophilized or hydrophobized). Typical examples include coated titanium dioxides such as T 805 titanium dioxide (Degussa) or Eusolex® T2000 (Merck). Suitable hydrophobic coating agents are chiefly silicones, more particularly trialkoxyoctylsilanes, preferably triethoxycaprylylsilanes or simethicones. Further preferred coating agents include aluminum oxides. A further preferred coating agent is silicon dioxide such as that commercially available from Merck KGaA under the name Eusolex T AVO. A further preferred coating agent is stearic acid. A further preferred coating agent is polyhydroxystearic acid. A further preferred coating agent is aluminum stearate. A particularly preferred inorganic UV filter is a titanium oxide hydrophobically coated with triethoxycaprylylsilane, available under the designation Titan M 265 from Kemira.

Compositions according to the present invention contain at least one inorganic filter substance in an amount of from 0.1 to 15 wt %, preferably 0.5 to 10 wt %, more preferably 1.0 to 5 wt %, and even more preferably 2.0 to 4.0 wt %, based on total weight of the composition.

In order further to improve the haptic properties of compositions according to the invention, the oil-in-water emulsions preferably contain arachyl alcohol and/or behenyl alcohol. Particularly preferred oil-in-water emulsions contain arachyl alcohol and/or behenyl alcohol in an amount of from 2.5 to 4.5 wt %, preferably 3 to 4 wt %, based on total weight of the composition.

Further preferred oil-in-water emulsions contain a mixture of dipotassium monocetyl phosphate and potassium dicetyl phosphate with hardened palm oil glycerides; furthermore Polysilicone-15, 4-tert-butyl-4′-methoxydibenzoylmethane, the sodium salt of 2-phenylbenzimidazole-5-sulfonic acid, arachyl alcohol and/or behenyl alcohol, in an amount of from 2.5 to 4.5 wt %, preferably 3 to 4 wt %, and having a pH at 20° C. from 6.0 to 8, preferably 6.5 to 7.5, more preferably 6.8 to 7.3.

Further preferred oil-in-water emulsions contain a mixture of dipotassium monocetyl phosphate and potassium dicetyl phosphate with hardened palm oil glycerides, furthermore Polysilicone-15, 4-tert-butyl-4′-methoxydibenzoylmethane, the sodium salt of 2-phenylbenzimidazole-5-sulfonic acid, arachyl alcohol and/or behenyl alcohol, in an amount of from 2.5 to 4.5 wt %, preferably 3 to 4 wt %, and having a pH at 20° C. from 6.0 to 8, preferably 6.5 to 7.5, more preferably 6.8 to 7.3, and are free of s-triazine derivatives according to the TRIAZINE SKELETON structural element.

In order to further improve the haptic properties and/or light protection factor of compositions according to the present invention, the compositions can preferably contain at least one particulate solid chosen from coloring, color-imparting, matting, or gloss-imparting pigments. Preferred pigments of this type can be inorganic or organic. Further preferred pigments have an arithmetically averaged particle diameter from 0.1 to 200 μm, preferably 0.5 to 100 μm, more preferably 1 to 50 μm, and even more preferably 2 to 30 μm. Particularly preferred inorganic pigments include the oxides of silicon, titanium, iron, zinc, zirconium, magnesium, cerium, and bismuth, as well as bismuth oxychloride, boron nitride, mica, fluorite, and water-insoluble pearlescent pigments which can be coated with at least one inorganic and/or organic compound. Useful pearlescent pigments are obtainable in numerous ways that are known per se. For example, other substrates in addition to mica can also be coated with further metal oxides (e.g., silica).

Dyes and pigments can be present both individually and in a mixture, and can be mutually coated with one another. Different color effects can generally be produced by way of differing coating thicknesses.

Further preferred pigments include colored and colorless pigments. Some of the pigments mentioned below also serve as UV absorbers. Particularly preferred colored pigments include iron oxides having the Color Index numbers CI 77491 (red iron oxide), CI 77492 (yellow iron oxide hydrate), CI 77499 (black iron oxide), CI 77891 (titanium dioxide) and carbon black.

A particularly preferred pigment is the commercial product SUNPMMA-S and SUNSIL Tin 30 available from the Sunjin Chemicals Company, having an arithmetically averaged particle diameter from 5 to 10 μm and 2 to 7 μm, respectively.

Preferred optional inorganic pigments are coated. Coating can be accomplished with the aid of inorganic and/or organic compounds. Inorganic pigments having an inorganic coating are particularly preferred. Particularly preferred pigments of this type include silicon dioxide particles coated with titanium dioxide and/or iron oxides. A particularly preferred pigment of this type is the commercial product Ronasphere® LDP of Merck KGaA. This is spherical pigment of silicon dioxide particles coated with titanium dioxide and iron oxide. Ronasphere® LDP has an arithmetically averaged particle diameter from 4 to 7 μm. Also preferred are inorganically coated mica pigments that exhibit no pearlescence. Further inorganically coated inorganic pigments include mica pigments coated with titanium dioxide at various layer thicknesses such as the products of the Timiron® series of Rona/Merck KGaA, particularly pigments of the Timiron® MP, Timiron® Super, Timiron® Starlight, and Timiron® Silk product lines. These products have average particle diameters from 5 to 60 μm, 10 to 60 μm, 10 to 125 μm, and 5 to 25 μm, respectively. Likewise preferred are mica particles having a coating of titanium dioxide and iron oxide such as the commercial products Timiron® MP-20, MP-24, MP-25, MP-28, MP-29, MP-60, and MP-65. Also preferred are mica particles coated with titanium dioxide and/or red and/or black iron oxide (e.g., the products of the Colorona® series). Further preferred pigments include mica pigments coated with silica gel (e.g., the commercial product Micronasphere® M). Further preferred pigments include inorganically coated inorganic pigments whose coating comprises a 0.1 to 1 wt % proportion of tin oxide.

Inorganic pigments coated with organic substances are also preferred. Examples include titanium dioxide pigments coated with aluminum stearate (e.g., the commercial product MT 100 T of the Tayca company), zinc oxide coated with dimethylpolysiloxane (dimethicone), boron nitride coated with dimethicone (Très BN® UHP 1106 of Carborundum), titanium dioxide coated with a mixture of dimethylpolysiloxane and silica gel (simethicone) and aluminum oxide hydrate (alumina) (Eusolex® T 2000 of Merck), and titanium dioxide or spherical polyalkylsesquisiloxane particles coated with octylsilanol (Aerosil® R972 of Degussa).

Further preferred compositions contain at least one coloring, color-imparting, matting, or gloss-imparting pigment in an amount of from 0.1 wt % to 30 wt %, preferably 0.5 to 15 wt %, more preferably 1.0 to 10 wt %, and even more preferably 2 to 5 wt %, based on total weight of the composition.

Further preferred particulate solids include polymethylsilsesquioxanes, obtainable by hydrolysis and condensation reactions from methyltrimethoxysilanes (in particular Aerosil® R 972 and Aerosil® 200 V of Evonik Degussa), which can be spherical and whose particle size distribution can be controlled by their manufacturing process. These particulate solids can be used both individually and in a mixture.

Total amount of the optional particulate solids is preferably 0.1 to 30 wt %, more preferably 0.5 to 15 wt %, even more preferably 1.0 to 10 wt %, and most preferably 2 to 4 wt %, based on total weight of the composition.

Compositions according to the present invention can also contain cosmetic adjuvants and other active substances typically used in such compositions (e.g., preservatives, preservation aids, bactericides, perfumes, dyes, thickening agents, moisturizing and/or moisture-retaining substances, fats, oil, waxes, alcohols, polyols, polymers, electrolytes, organic solvents, or silicone derivatives).

Useful preservatives include formaldehyde releasers (e.g., DMDM hydantoin), iodopropyl butylcarbamates, parabens, phenoxyethanol, ethanol, benzoic acid, and many similar others.

The preservation system furthermore includes preservation aids such as octoxyglycerol, 1,2-pentanediol, 1,5-pentanediol, 1,2-hexanediol, 1,6-hexanediol, 1,2-heptanediol, 1,2-octanediol, 1,8-octanediol, 1,2-nonanediol, 1,2-decanediol, 1,10-decanediol, 1,2-undecanediol, 1,2-dodecanediol, 1,2-tridecanediol, or 1,2-tetradecanediol.

Particularly preferred compositions include antioxidants used as additives or active substances. Preferably, the compositions contain at least one antioxidant. All antioxidants suitable for cosmetic and/or dermatological applications, in particular tocopherol, tocopheryl acetate, or dimethylmethoxy chromanol, obtainable from Lipotec under the commercial name Lipochroman-6, can optionally be used as favorable antioxidants.

In additionally advantageous fashion, active substance or substances include catechin and gallic acid esters of catechins, and aqueous or organic extracts from plants or plant parts having a content of catechins or gallic acid esters of catechins, for example, the leaves of the Theaceae plant family, particularly the species Camellia sinensis (green tea). Typical ingredients thereof (such as polyphenols or catechins, caffeine, vitamins, sugars, minerals, amino acids, lipids) are particularly advantageous.

Catechins represent a group of compounds referred to as hydrogenated flavones or anthocyanidines and represent derivatives of “catechin” (catechol, 3,3′,4′,5,7-flavanepentaol, 2-(3,4-dihydroxyphenyl)-chromane-3,5,7-triol). Epicatechin ((2R,3R)-3,3′,4′,5,7-flavanepentaol) is also an advantageous active substance for purposes of the present invention.

Also advantageous are plant extracts having a catechin content, particularly extracts of green tea such as extracts from leaves of plants of the Camellia species, more particularly the tea varieties Camellia sinensis, C. assamica, C. taliensis, C. irrawadiensis, and crosses between these, as well as, for example, Camellia japonica. Preferred active substances are furthermore polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

In a further preferred embodiment, compositions according to the present invention contain at least one flavonoid or at least one flavonoid-rich plant extract.

Useful flavonoids include the glycosides of flavones, of flavanones, of 3-hydroxyflavones (flavonoles), of aurones, and of isoflavones. Particularly preferred flavonoids include naringin (aurantiin, naringenin 7-rhamnoglucoside), α-glucosylrutin, α-glucosylmyricetin, α-glucosylisoquercetin, α-glucosylquercetin, dihydroquercetin (taxifolin), hesperidin (3′,5,7-trihydroxy-4′-methoxyflavanone 7-rhamnoglucoside, hesperitin 7-O-rhamnoglucoside), neohesperidin, rutin (3,3′,4′,5,7-pentahydroxyflavone 3-rhamnoglucoside, quercetin 3-rhamnoglucoside), troxerutin (3,5-dihydroxy-3′,4′,7-tris(2-hydroxyethoxy)flavone-3-(6-O-(6-deoxy-α-L-mannopyranosyl) β-D-glucopyranoside)), monoxerutin (3,3′,4′,5-tetrahydroxy-7-(2-hydroxyethoxy)flavone-3-(6-O-(6-deoxy-α-L-mannopyranosyl) β-D-glucopyranoside)), diosmin (3′,4′,7-trihydroxy-5-methoxyflavanone 7-rhamnoglucoside), eriodictin, and apigenin 7-glucoside (4′,5,7-trihydroxyflavone 7-glucoside). Flavonoids most preferred according to the present invention include α-glucosylrutin, naringin, and apigenin 7-glucoside.

Also preferred are biflavonoids constructed from two flavonoid units, for example, those occurring in ginkgo species. Further preferred flavonoids are the chalcones, especially phloricin, hesperidin methyl chalcone, and neohesperidin dihydrochalcone.

Particularly preferred compositions contain at least one flavonoid in an amount of from 0.0001 to 1 wt %, preferably 0.0005 to 0.5 wt %, and more preferably 0.001 to 0.1 wt %, based on flavonoid active substance in the entire cosmetic composition.

In a further preferred embodiment, compositions according to the present invention contain at least one ubiquinone or one ubiquinol, or derivatives thereof. Ubiquinols are the reduced form of ubiquinones. Ubiquinones preferred according to the present invention have the formula (UBI-I)—

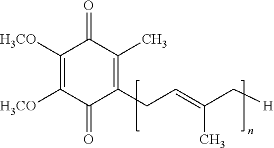

wherein n=6, 7, 8, 9, or 10.

The ubiquinone of formula (UBI-I) where n=10, also known as coenzyme Q10, is particularly preferred.

Compositions particularly preferred according to the present invention contain at least one ubiquinone, ubiquinol, or a derivative thereof, in an amount of from 0.0001 to 1 wt %, preferably 0.001 to 0.5 wt %, and more preferably 0.005 to 0.1 wt %, based on total weight of the composition.

In a further preferred embodiment, the compositions contain silymarin. Silymarin is an active-substance concentrate, formerly regarded as a uniform substance, from the fruits of the milk thistle (Silybum marianum). The main components of silymarin are silybin (silymarin I), silychristin (silymarin II), and silydianin, which belong to the flavanolignans.

Particularly preferred compositions contain silymarin in an amount of from 0.00001 to 1 wt %, preferably 0.0001 to 0.01 wt %, and more preferably 0.005 to 0.1 wt %, based on total weight of the composition.

In a further preferred embodiment, the compositions contain at least one naturally occurring xanthine derivative chosen from caffeine, theophylline, theobromine, and aminophylline. According to the present invention, naturally occurring xanthine derivatives are present in amounts of from 0.0001 to 1 wt %, preferably 0.001 to 0.5 wt %, and more preferably 0.005 to 0.1 wt %, based on total weight of the composition.

In a further preferred embodiment, the compositions contain ectoin. Ectoin is the common name for 2-methyl-1,4,5,6-tetrahydropyrimidine-4-carboxylate. According to the present invention, ectoin is present in an amount of from 0.0001 to 1 wt %, preferably 0.001 to 0.5 wt %, and more preferably 0.005 to 0.01 wt %, based on total weight of the composition.

In a further preferred embodiment, the compositions contain at least one natural betaine compound. Preferred natural betaine compounds include naturally occurring compounds having the atomic grouping R₃N⁺—CH₂—X—COO⁻ per IUPAC rule C-816.1. Betaine surfactants (synthetic) are not included in betaine compounds used according to the present invention, and neither are other zwitterionic compounds in which the positive charge is present on N or P and the negative charge formally on O, S, B, or C, but which do not conform to IUPAC Rule C-816.1 Betaine compounds preferred according to the present invention include betaine (Me₃N⁺—CH₂—COO⁻), carnitine (Me₃N⁺—CH₂—CHOH—CH₂—COO⁻), wherein Me=methyl, and ergothioneine=2-mercapto-N^{α}N^{α}N^{α}-trimethyl-L-histidinium betaine of formula (BETA-II)—

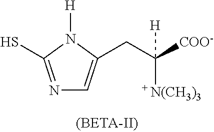

Derivatives of natural betaine compounds that can be used can be synthetic.

Salts and/or esters of natural betaine compounds can also preferably be used according to the present invention. Particularly preferred derivatives include carnitine tartrate, propionylcarnitine, and particularly acetylcarnitine. Both enantiomers (D and L form) can be used advantageously for purposes of the present invention. It may also be advantageous to use any enantiomer mixtures, for example, a racemate of the D and L form.

Preferred compositions according to the present invention contain at least one natural betaine compound in an amount of from 0.01 to 5 wt %, preferably 0.1 to 3 wt %, more preferably 0.2 to 1 wt %, and even more preferably 0.3 to 0.5 wt %, based on total weight of the composition.

Further compositions preferred according to the present invention contain at least one conditioning active substance. “Conditioning active substances” according to the present invention include those substances that are absorbed onto keratinic materials, in particular onto the skin, and improve the physical and sensory properties of both the skin and the product.

Conditioning agents smooth the uppermost layer of the skin and make it soft and supple. The skin feel of the entire product can be adjusted by selection of the conditioning agents (greasy or less greasy, fast- or slow-spreading, rapidly or slowly absorbed into the skin, and so forth).

Preferred conditioning active substances include fatty substances, in particular plant oils such as sunflower oil, olive oil, soybean oil, rapeseed oil, almond oil, jojoba oil, orange oil, wheat germ oil, peach kernel oil, and the liquid components of coconut oil, lanolin and derivatives thereof, liquid paraffin oils, isoparaffin oils, and synthetic hydrocarbon oils; di-n-alkyl ethers having 12 to 36 carbon atoms (e.g., di-n-octyl ether and n-hexyl-n-octyl ether); fatty acids, particularly linear and/or branched, saturated and/or unsaturated C₈₋₃₀ fatty acids; fatty alcohols, particularly saturated, mono- or polyunsaturated, branched or unbranched fatty alcohols having 4 to 30 carbon atoms, which can be ethoxylated with 1 to 75, preferably 5 to 20 ethylene oxide units and/or propoxylated with 3 to 30, preferably 9 to 14 propylene oxide units; ester oils (i.e., esters of C₆₋₃₀ fatty acids with C₂₋₃₀ fatty alcohols); hydroxycarboxylic acid alkyl esters, dicarboxylic acid esters such as di-n-butyl adipate, and diol esters such as ethylene glycol dioleate or propylene glycol di(2-ethylhexanoate), symmetrical, asymmetrical, or cyclic esters of carboxylic acid with fatty alcohols (e.g., glycerol carbonate or dicaprylyl carbonate (Cetiol® CC)); mono-, di-, and trifatty acid esters of saturated and/or unsaturated linear and/or branched fatty acids with glycerol, which can be ethoxylated with 1 to 10, preferably 7 to 9 ethylene oxide units (e.g., PEG-7 Glyceryl Cocoate); waxes, in particular insect waxes, plant waxes, fruit waxes, ozocerite, microcrystalline waxes, ceresin, paraffin waxes; triglycerides of saturated and optionally hydroxylated C₁₆₋₃₀ fatty acids (e.g., hardened triglyceride fats); phospholipids such as soy lecithin, egg lecithin, and cephalins; silicone compounds chosen from decamethylcyclopentasiloxane, dodecamethylcyclohexasiloxane; and silicone polymers which can be crosslinked if desired (e.g., polydialkylsiloxanes, polyalkylarylsiloxanes, ethoxylated and/or propoxylated polydialkylsiloxanes having the former INCI designation Dimethicone Copolyol, as well as polydialkylsiloxanes that contain amine groups and/or hydroxy groups, preferably substances having the INCI designations Dimethiconol, Amodimethicone, or Trimethylsilylamodimethicone).

The amount of fatty substances used is preferably 0.1 to 99 wt %, more preferably 2 to 50 wt %, and even more preferably 5 to 20 wt %, based on total weight of the composition.

Cosmetic or dermatological compositions according to the present invention can exist in the form of a liquid, flowable, or solid oil-in-water emulsion.

Further suitable additives include thickening agents such as anionic polymers of acrylic acid, methacrylic acid, crotonic acid, maleic acid anhydride, and 2-acrylamido-2-methylpropane-sulfonic acid, in which the acid groups can be present entirely or partly as a sodium, potassium, ammonium, mono- or triethanolamine salt. At least one nonionic monomer can be present. Preferred nonionic monomers include acrylamide, methacrylamide, acrylic acid esters, methacrylic acid esters, vinylpyrrolidone, vinyl ethers, and vinyl esters. Preferred anionic copolymers include acrylic acid-acrylamide copolymers, particularly polyacrylamide copolymers containing sulfonic acid groups. The copolymers can also be present in crosslinked form. Suitable commercial products are Sepigel® 305, Simulgel® 600, Simulgel® NS, Simulgel® EPG, and Simulgel® EG from the SEPPIC company. Further particularly preferred anionic homo- and copolymers include uncrosslinked and crosslinked polyacrylic acids. Such compounds are, for example, the Carbopol® commercial products. A particularly preferred anionic copolymer contains, as monomer, 80 to 98% of an unsaturated C₃₋₆ carboxylic acid, substituted if desired, or its anhydride, as well as 2 to 20% acrylic acid esters, substituted if desired, of saturated C₁₀₋₃₀ carboxylic acids, so that the copolymer can be crosslinked with the aforesaid crosslinking agents. Corresponding commercial products are Pemulen® and the Carbopol® grades 954, 980, 1342 and ETD 2020 (from Lubrizol).

Suitable nonionic polymers include polyvinyl alcohols that can be partly saponified (e.g., the Mowiol® commercial products, as well as vinylpyrrolidone/vinyl ester copolymers and polyvinylpyrrolidones that are marketed, for example, under the trademark Luviskol® (BASF)).

Further preferred additives include solvents such as ethanol, isopropanol, ethylene glycol, propylene glycol, propylene glycol monoethyl ether, glycerol, and diethylene glycol, perfume oils, substances for adjusting pH, complexing agents such as EDTA, NTA, β-alaninediacetic acid, and phosphonic acids, propellants such as propane-butane mixtures, pentane, isopentane, isobutane, N₂O, dimethyl ether, CO₂, and air.

In a further preferred embodiment, the compositions are packaged in a spray or pump dispenser.

The present invention is also directed towards use of the above-defined compositions for protecting skin and hair from the damaging effects of UV radiation, particularly UV-A and UV-B radiation, more particularly solar radiation.

Regarding further preferred embodiments of the uses according to the present invention, statements concerning compositions according to the present invention apply mutatis mutandis.

The present invention also provides for a non-therapeutic cosmetic method for protecting skin and hair from the damaging effects of UV-radiation, particularly UV-A and UV-B radiation, more particularly solar radiation, in which a composition according to the present invention is applied in an amount effective in protecting the skin onto the skin.

Regarding further preferred embodiments of the method according to the present invention, statements concerning compositions according to the present invention apply mutatis mutandis.

### EXAMPLES

The Examples below (numbers 1 to 4 according to the present invention) are intended to illustrate compositions according to the present invention without limiting it thereto. Numerical values in the Examples denote percentages by weight, based on total weight of the composition.

| | No. 1 | No. 2 | No. 3 | No. 4 | Comparison |
|---|---|---|---|---|---|
| Emulsiphos 677660 | 2.5 | 3 | 3 | 3 | 3 |
| Dibutyl adipate | 5 | 5 | 5 | 5 | 5 |
| Caprylic/Capric Triglycerides | 3.5 | 3.5 | 3.5 | 3.5 | 3.5 |
| Safflower oil, refined | 2 | 2 | 2 | 2 | 2 |
| Behenyl alcohol | 3 | 3 | 3 | 3 | 3 |
| Diisopropyl adipate | 1 | — | — | — | — |
| 2-Propylheptyl octanoate | — | 1 | 1 | 1 | 1 |
| Vitamin E acetate | 0.5 | 0.5 | 0.5 | 0.5 | 0.5 |
| Controx KS | 0.05 | 0.05 | 0.05 | 0.05 | 0.05 |
| Beeswax | 0.2 | 0.2 | 0.2 | 0.2 | 0.2 |
| Cosmedia SP | 0.5 | 0.5 | 0.5 | 0.5 | 0.5 |
| Butylmethoxydibenzoylmethane | 3 | 3 | 3 | 3 | 4 |
| Parsol SLX | 2 | 2 | 2 | 2 | 4 |
| Octocrylene | 4 | 3 | 2 | 2 | — |
| Tetrasodium EDTA* | 0.1 | 0.1 | 0.1 | 0.1 | 0.1 |
| Ethylparaben | 0.2 | 0.2 | 0.2 | 0.2 | 0.2 |
| Hexanediol-1,6 | 6 | 6 | 6 | 6 | 6 |
| Glycerol 86% | 4.5 | 4.5 | 4.5 | 4.5 | 4.5 |
| Methylparaben | 0.2 | 0.2 | 0.2 | 0.2 | 0.2 |
| Carbopol ETD 2020 | 0.2 | 0.2 | 0.2 | 0.2 | 0.2 |
| Aristoflex AVC | — | — | 0.6 | 0.6 | — |
| Cosmedia Silc | 2 | 2 | 2 | 2 | 2 |
| PhytoCellTec Malus domestica | — | 4 | 4 | 4 | — |
| Polyol prepolymer-2 | — | 1 | 1 | 1 | — |
| Matrixyl 3000 PEG-free | — | 5 | 5 | 5 | — |
| DSH C N | — | 6 | 6 | 6 | — |
| Vitamin A palmitate | — | 0.3 | 0.3 | 0.3 | — |
| 1.7 m · IU/g | | | | | |
| Perfume | — | 0.3 | 0.3 | 0.3 | — |
| Phenoxyethanol | 0.4 | 0.4 | 0.4 | 0.4 | 0.4 |
| Lipochroman-6 | — | 0.1 | 0.1 | 0.1 | — |
| Sodium hydroxide, pellets | 0.2394 | 0.251 | 0.277 | 0.371 | 0.440 |
| 2-Amino-2-methylpropan-1-ol | 1 | 1 | 1 | 1 | 1 |
| Neo Heliopan Hydro | 2 | 2 | 2 | 2 | 3 |
| Neo Heliopan AP | 1 | 1 | 1 | 1 | — |
| Water, deionized | to 100 | to 100 | to 100 | to 100 | to 100 |
| pH | 7.3 | 7.3 | 7.3 | 7.3 | 7.3 |
| SPF (in vivo) | >50 | 47.6 ± 9.6 | 43.4 ± 4.9 | 41.5 ± 7.6 | 34.1 ± 9.2 |
| | | (n = 5) | (n = 5) | (n = 5) | |
| SPF (in vivo)/UVAPF | n.c. | 2.6 | 2.5 | 2.1 | n.c. |

*Powder, incorporated into the oil phase.

Further comparison formulations (having a total of at least 12 wt % UV filters; without octocrylene)—

| | | |
|---|---|---|
| Emulsiphos 677660 | 3 | 3 |
| Dibutyl adipate | 5 | 5 |
| Caprylic/Capric Triglycerides | 3.5 | 3.5 |
| Safflower oil, refined | 2 | 2 |
| Behenyl alcohol | 3 | 3 |
| Diisopropyl adipate | — | — |
| 2-Propylheptyl octanoate | 1 | 1 |
| Vitamin E acetate | 0.5 | 0.5 |
| Controx KS | 0.05 | 0.05 |
| Beeswax | 0.2 | 0.2 |
| Cosmedia SP | 0.5 | 0.5 |
| Butylmethoxydibenzoylmethane | 4 | 5 |
| Parsol SLX | 4 | 5 |
| Solaveil CT 100 | 5 | — |
| Octocrylene | — | — |
| Tetrasodium EDTA* | 0.1 | 0.1 |
| Ethylparaben | 0.2 | 0.2 |
| Hexanediol-1,6 | 6 | 6 |
| Glycerol 86% | 4.5 | 4.5 |
| Methylparaben | 0.2 | 0.2 |
| Carbopol ETD 2020 | 0.2 | 0.2 |
| Aristoflex AVC | — | — |
| Cosmedia Silc | 2 | 2 |
| PhytoCellTec Malus domestica | — | — |
| Polyol prepolymer-2 | — | — |
| Matrixyl 3000 PEG-free | — | — |
| DSH C N | — | — |
| Vitamin A palmitate 1.7 m · IU/g | — | — |
| Perfume | — | — |
| Phenoxyethanol | 0.4 | 0.4 |
| Lipochroman-6 | — | — |
| Sodium hydroxide, pellets | 0.435 | 0.556 |
| 2-Amino-2-methylpropan-1-ol | 1 | 1 |
| Neo Heliopan Hydro | 3 | 4 |
| Neo Heliopan AP | — | — |
| Water, deionized | ad 100 | ad 100 |
| pH | | |
| | 7.3 | 7.3 |
| SPF (in vivo) | 21.0 ± 5.0 | 34.8 ± 5.7 |
| SPF (in vivo)/UVAPF | n.c. | n.c. |

List of raw materials used—

| Commercial name | INCI | Manufacturer |
|---|---|---|
| Carbopol ETD 2020 | Acrylates/C10-30 Alkyl Acrylate Crosspolymer | Lubrizol |
| Controx KS | Tocopherol, Hydrogenated Palm Glycerides | Cognis |
| | Citrate | |
| Cosmedia Silc | Silica | Cognis |
| Cosmedia SP | Sodium Polyacrylate | Cognis |
| DSH C N | Dimethylsilanol Hyaluronate (0.5 wt % aqueous | Exsymol |
| | solution) | |
| Emulsiphos 677660 | Potassium Cetyl Phosphate, Hydrogenated Palm | Symrise |
| | Glycerides | |
| Lipochroman-6 | Dimethylmethoxy Chromanol | Lipotec |
| Matrixyl 3000 PEG free | Glycerin, Aqua (Water), Butylene Glycol, | Sederma |
| | Carbomer, Coco Glucoside, Palmitoyl | |
| | Oligopeptide, Palmitoyl Tetrapeptide-7 | |
| Neo Heliopan AP | Disodium Phenyl Dibenzimidazole Tetrasulfonate | Symrise |
| Neo Heliopan Hydro | Phenylbenzimidazole Sulfonic Acid | Symrise |
| Parsol SLX | Polysilicone-15 | DSM |
| PhytoCellTec Malus Domestica | Aqua (Water), Malus Domestica Fruit Cell | Mibelle AG |
| | Culture Extract, Xanthan Gum, Glycerin, | |
| | Lecithin, Phenoxyethanol | |
| Polyol prepolymer-2 | PPG-12/SMDI COPOLYMER | Sederma |
| Solaveil CT 100 = 45 wt % TiO₂ (= | C12-15 Alkyl Benzoate, Titanium Dioxide, | Croda |
| inorganic UV filter) dispersed in C12-15 | Aluminum Stearate, Polyhydroxystearic Acid, | |
| Alkyl Benzoate | Alumina | |

We claim:

1. Cosmetic oil-in-water composition comprising:
(a) UV filter 4-(tert-butyl)-4′-methoxydibenzoylmethane,
(b) UV filter Polysilicone-15,
(c) UV filter 2-cyano-3,3-phenylcinnamic acid 2-ethylhexyl ester,
(d) at least one water-soluble UV filter chosen from at least one derivative of benzimidazole sulfonic acid,
(e) at least one neutralization agent for the water-soluble UV filter chosen from amino-C₁ to C₆ alkanols and/or amino-C₁ to C₆ alkanediols, in particular selected from 2-amino-2-methylpropan-1-ol, 2-amino-2-methylpropane-1,3-diol, 2-aminopropan-1-ol, 3-aminopropan-1-ol, 1-aminopropan-2-ol (MIPA), or 2-amino-2-(hydroxymethyl)propane-1,3-diol (TRIS),
(f) at least one salt of C₁₂₋₂₀ alkyl phosphate as an anionic oil-in-water emulsifier, and
(g) at least one C₁₄₋₂₀ mono- or diacyl glyceride,
wherein the composition has a pH of 6.0 to 8.0 at 20° C.

2. Cosmetic composition according to claim 1, wherein the at least one derivative of benzimidazole sulfonic acid of the at least one water-soluble organic UV filter is chosen from phenylene-1,4-bis-(2-benzimidazyl)-3,3′-5,5′-tetrasulfonic acid, 2-phenylbenzimidazole-5-sulfonic acid, and the salts thereof.

3. Cosmetic composition according to claim 1 further comprising amino acids in an amount of from 0 to 0.1 wt %, based on total weight of the composition.

4. Cosmetic composition according to claim 1, wherein the at least one salt of C₁₂₋₂₀ alkyl phosphate and the at least one glyceride are present in an amount of from 0.5 wt % to 10 wt %, based on total weight of the composition.

5. Cosmetic composition according to claim 1, wherein the 4-(tert-butyl)-4′-methoxydibenzoylmethane is present in an amount of from 0.5 wt % to 20 wt %, based on total weight of the composition.

6. Cosmetic composition according to claim 1, wherein the Polysilicone-15 is present in an amount of from 0.5 wt % to 10 wt %, based on total weight of the composition.

7. Cosmetic composition according to claim 1, wherein the 2-cyano-3,3-phenylcinnamic acid 2-ethylhexyl ester is present in an amount of from 0.5 wt % to 15 wt %, based on total weight of the composition.

8. Cosmetic composition according to claim 1, wherein the UV filters (a), (b), (c) and (d) are present in a weight ratio (a):(b):(c):(d) to one another of (2.7 to 3.3):(1.8 to 2.2):(1.8 to 3.3):(2.7 to 3.3).

9. Cosmetic composition according to claim 1 free of UV filter substances based on s-triazine derivatives comprising the following structural element—

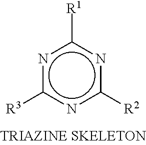

10. Non-therapeutic cosmetic method for protecting skin and hair from the damaging effects of UV-radiation comprising applying a composition according to claim 1 in an effective amount onto the skin.
